(19) United States
(12) Patent Application Publication
(10) Pub. No.: US 2002/0012667 A1
(43) Pub. Date: Jan. 31, 2002

(54) Method for preventing allograft rejection

(76) Inventor: John McMichael, Delanson, NY (US)

Correspondence Address: MARSHALL, O'TOOLE, GERSTEIN, MURRAY & BORUN, 6300 SEARS TOWER, 233 SOUTH WACKER DRIVE, CHICAGO, IL, 60606-6402, US

(21) Appl. No.: 09/834,450

(22) Filed: Apr. 13, 2001

Related U.S. Application Data
(60) Provisional application No. 60/204,631, filed on May 16, 2000.

(51) Int. Cl. A61K 39/00

(52) U.S. Cl. 424/184.1

(57) ABSTRACT

The present invention provides a method of preventing allograft rejection in a transplant recipient comprising the step of administering to the recipient an antigenic preparation presenting antigens characteristic of the allograft in an amount effective to neutralize the immune response to the allograft.

This application claims the benefit of U.S. Provisional Patent Application Serial No. 60/204,631, filed May 16, 2000, the disclosure of which is hereby incorporated by reference.

### BACKGROUND OF THE INVENTION

The present invention relates to organ and tissue transplantation and specifically to transplantation of allografts having the potential for host rejection.

The most significant limitation on the success of allographic tissue and organ transplantation is the immunological rejection of the transplanted tissue by the host. The rejection of tissue (the term as used herein includes organs) transplants involves both cell-mediated and antibody-mediated responses which are targeted on the HLA antigens of the graft. The classic acute rejection, which occurs within 10 to 14 days in non-immunosuppressed recipients, is largely the result of a T-cell mediated hypersensitivity reaction. The generation of cytotoxic T lymphocytes (CTLs), e.g., T-helper cells and “pre-killer T cells” which bear receptors for foreign HLA antigens differentiate into mature CTLs which lyse the grafted tissue. Although the helper cells cannot differentiate into killer cells, they are necessary for efficient generation of cytotoxic cells. In addition to the specific cytotoxic T cells, sensitization also leads to the generation of lymphokine secreting T cells as in the classic delayed hypersensitivity reaction, which leads to local accumulation of macrophages which also take part in graft destruction.

In addition to T cell-mediated rejection, there also exists antibody-mediated rejection. Hyperacute rejection can take place where a subject is presensitized and has formed antibodies against donor tissue. Such can occur in the case of multiparous women who develop anti-HLA antibodies against paternal antigens shed from the fetus. Prior blood transfusions from HLA non-identical donors can also lead to presensitization.

Because host rejection of grafts is linked to genetically determined immunologic markers, efforts are made to match potential donor tissue with recipients. In addition to ABO and other blood group antigens, HLA antigens play a major role in determining immunologic identity. HLA-A and HLA-B markers are expressed on the cell membranes of all nucleated cells and are coded by a cluster of genes known as the major histocompatibility complex. While a graft from an identical twin would be preferred because no immunosuppressive therapy would be required, such donors are rarely available. In general an ABO compatible donor who has at least one matching HLA antigen would have a greater probability of survival than a graft from a donor having no matching antigens. Among other siblings, only one in four is likely to have two HLA haplotypes in common, one half would have one HLA haplotype, and one in four would share no matching antigens. Parent-offspring matches will always share only one haplotype.

Donors from the general population are screened by HLA-A, HLA-B and sometimes by other groups such as HLA-DR for compatibility, but except in the case where the subject suffers from severe combined immunodeficiency disease, immunosuppressive therapy is required to prevent host rejection of the transplant. Immunosuppressive therapies include those such as administration of corticosteroids, such as prednisone, administration of cytotoxic drugs, radiation therapy with X-rays, antilymphocyte globulins and antithymocyte globulins, cyclosporine and newer experimental agents. Each of these immunosuppressive therapies is accompanied by significant adverse side-effects including cytotoxic effects and is subject to unwanted drug interactions. Perhaps even more significantly, immunosuppressive therapy renders the recipient vulnerable to opportunistic infections and increases the chances of occurrence of neoplastic disease. Accordingly, there exists a desire in the art for alternative treatments which could eliminate or reduce the extent of immunosuppressive therapy.

### SUMMARY OF THE INVENTION

The present invention relates to the discovery that tolerance to transplantation of allografts can be promoted in a transplant recipient by administering to that recipient an antigenic preparation presenting antigens characteristic of the allograft in an amount effective to neutralize the immune response.

Specifically, the invention provides a method of preventing allograft rejection in a transplant recipient comprising the step of administering to the recipient an antigenic preparation presenting antigens characteristic of the allograft in an amount effective to neutralize the immune response during and optionally immediately before the transplantation event. Moreover, it is preferred that the antigenic preparation presenting antigens characteristic of the allograft continue to be administered to the transplant recipient for a period of from several days to a week after the transplantation event.

Dosages of antigenic preparations useful according to the invention may be determined empirically by those skilled in the art but typically range from 10⁻⁸ to 10³ grams of antigenic material per dose with dosages of 10⁻⁴ to 10⁻¹ grams per dose being preferred. According to one method for determining dosages for practice of the invention, a useful dosage may be determined as an amount which is a five-fold dilution below the highest dilution that elicits a positive wheal/flare response to a skin test in which the antigenic preparation is intradermally administered to the skin of the transplant recipient. The antigenic preparation may be administered in multiple dosages the day of the transplantation event but a single daily dosage can be effective within days of the transplantation event.

The antigenic preparation presents antigens characteristic of the donor tissue in order to promote tolerance to those antigens by the host. While the antigenic preparation preferably comprises donor tissue which has been mechanically homogenized, alternative means of producing such preparations would be apparent to those of skill in the art. Such methods include but are not limited to those wherein antigens from sources other than the donor tissue, sonicated tissue and the like are combined to replicate the antigenicity of the donor tissue.

Preferred means of administration of the antigenic preparation include injection including (intravenous, intramuscular and subcutaneous), sublingual administration, oral administration and other means of administration known to those of skill in the art.

While the method of the invention maybe used alone to promote tolerance of the recipient to the allograft, it is contemplated that practice of the method will be particularly useful in combination with additional immunosuppressive therapy including conventional immunosuppressive therapy such as cyclosporine treatment and the like.

It is contemplated that the method of the invention will be useful with allografts of all types with particular utility wherein the allograft is a skin graft or a graft of pancreatic beta-cells. As used herein, the term “allograft” is defined broadly as including the living cells of a donor and includes cases in which the allograft is a transfusion of blood or serum.

### DETAILED DESCRIPTION

The invention is directed to the discovery that tolerance to the transplantation of allografts can be promoted in a transplant recipient by administering to that recipient an antigenic preparation presenting antigens characteristic of the allograft in a defined amount effective to neutralize the immune response of the host to the allograft.

The following examples are presented to more clearly illustrate the invention. Example 1 relates to promotion of tolerance in recipients of allografts which are blood transfusions in a rabbit model. Example 2 relates to promotion of tolerance in bovine recipients of equine erythrocytes. Example 3 relates to transplantation of rat pancreatic beta-cells into other rats. Example 4 relates to transplantation of canine pancreatic beta-cells into other dogs. Example 5 relates to transplantation of a skin allograft.

### EXAMPLE 1

According to this example, the therapeutic methods of the invention were evaluated in a model in which shock dosages of equine erythrocytes (red blood cells) were transfused into rabbits that were presensitized to the erythrocytes.

Whole blood was collected from the jugular vein of a Belgian horse. One liter of blood was withdrawn from the horse with a 14 gauge needle connected to a blood collection bag containing acid citrate dextrose (ratio of acid citrate dextrose to blood was 1:9). The erythrocytes were isolated according to a method in which whole blood was centrifuged at 1,000×g for 10 minutes. The supernatant plasma and the buffy coat were removed and discarded. The erythrocytes were washed using a volume of sterile saline (0.9%NaCl) equal to that of the plasma removed. The erythrocyte suspension was centrifuged at 1000×g for 10 minutes. The supernatant was removed and discarded and the wash procedure was repeated. The erythrocytes were resuspended in sterile saline and acid citrate dextrose was added in the same ratio as described above. The erythrocytes were placed at 4° C. until later use.

Transfusion of the equine erythrocytes was carried out according to a procedure in which erythrocytes were removed from 4° C. and slowly warmed to 37° C. Recipient rabbits were placed in a restrainer. The marginal ear vein was catheterized, and the catheter was flushed with sterile saline. A sensitization dose of up to 60 ml of erythrocytes was transfused over 25 minutes. Upon completion of the transfusion, the catheter was removed and the rabbit evaluated. After two weeks, a shock dose equal to that of the sensitization dose was administered.

In order to determine the therapeutic dosage, a series of five-fold dilutions of washed erythrocytes was made using sterile water. Starting with the lowest dilution, 0.02 ml was injected intradermally. A positive result was characterized by a wheal/flare response. The therapeutic dose was defined as the five-fold dilution below the highest dilution that elicited a positive result. A 0.2 ml dosage was administered to the rabbit during and after the shock dose.

A total of 13 rabbits were given both a sensitization dose and a shock dose. No severe adverse reactions were observed after administration of the sensitization dose. Two weeks later a skin test was performed as described above. The therapeutic dose was determined to be a 1:25 dilution of the washed erythrocytes.

The 13 rabbits were broken into two groups after the sensitization dose. Five rabbits were not given the therapeutic after administration of the shock dose. All five of those rabbits succumbed to an anaphylactic-type reaction characterized by respiratory distress, cyanosis and convulsions. Four of those rabbits died within an hour of receiving the shock dose. The other rabbit died 8 hours after administration of the shock dose.

The 8 remaining rabbits were given the 1:25 dilution prior to and during administration of the shock dose. Two of these rabbits expired within 12 hours of receiving the transfusion. The other 6 rabbits survived the transfusion and were given a 1:25 dilution once a day for a week. They were monitored for a two month period with no adverse reactions observed.

There was a significant difference in the reactions observed between non-medicated and medicated rabbits during the second transfusion. All of the non-medicated rabbits expired shortly after receiving the shock dose, whereas 6 of 8 of the medicated rabbits survived for more than 2 months after receiving the second transfusion. While it is not known whether the therapeutic dose allows for acceptance of the incompatible erythrocytes or whether it may act in a secondary fashion and interfere with certain aspects of the anaphylactic reaction, these results suggest that the therapeutic dose is protecting the medicated rabbits from the anaphylactic reaction observed in non-medicated rabbits.

### EXAMPLE 2

According to this example, the method of the invention is evaluated in treating the acute reaction caused by the transfusion of incompatible equine erythrocytes into a bovine model. The acute reaction is well characterized at the clinical and cellular levels and this example is directed to monitoring and comparing the clinical and cellular changes between treated and untreated animals.

Clinically the transfusion reaction consists of fever, chills, dyspnea, hypotension, shock, renal failure, and death. The reaction at the cellular level is characterized by a hemolytic reaction. It is caused by the formation of antigen-antibody complexes on the erythrocyte membrane. These complexes, in turn, activate the complement cascade, which leads to intravascular hemolysis, and the release of histamine and serotonin from mast cells. The release of histamine acts as a stimulant for gastric secretion and contraction of bronchial smooth muscle, while serotonin acts as a vasoconstrictor. The subsequent destruction of incompatible erythrocytes can cause elevated levels of hemoglobin, alkaline phosphatase, and eventually bilirubin in the plasma.

Whole blood was collected from the jugular vein of a Belgian horse according to the method of example 1 and stored at 4° C. for later use. Transfusion of the erythrocytes was carried out according to a method in which they were removed from 4° C. and slowly warmed to 37° C. The jugular vein of recipient calves was catheterized, and the catheter was flushed with sterile saline. A sensitization dose of 500 ml of erythrocytes was administered. During and after the transfusion the calves were evaluated, and blood samples were taken. If the calf survived administration of the sensitization dose, a shock dose equal to that of the sensitization dose was administered two weeks later.

The therapeutic dosage was determined according to a method in which a series of five-fold dilutions of washed erythrocytes was made using sterile water. Starting with the lowest dilution, 0.02 ml was injected intradermally. A positive result was characterized by a wheal/flare response. The therapeutic dose was defined as the five-fold dilution below the highest dilution that elicited a positive result. 0.2 ml of the therapeutic dose was given to the calf during and after the shock dose.

In the fourteen calves used for this experiment, nearly all of the clinical symptoms described above were observed upon administration of the shock dosages. A temperature change was characteristically noted between two and four hours post transfusion. Of the temperatures taken, four calves had a fever of2°-3° F. above normal, and seven calves showed a temperature decrease of 2°-5° F. All calves experienced respiratory difficulty. This was generally characterized by an initial coughing period within the first two minutes of transfusion which led to a significant increase in respiration. Dyspnea often followed with dilated nostrils and marked cyanosis. Shock was observed in several calves with cyanosis. This was characterized by unusual kicking and convulsing, shallow breathing, and fecal incontinence.

Elevated levels of hemoglobin, alkaline phosphatase, and bilirubin were found in the blood that was collected from several calves. Absorbency readings produced an optical density of over 3.00 within an hour post transfusion, while initial readings gave values below 1.00. Absorbency levels did not begin to decrease until 5-6 hours post transfusion. Several calves showed a significant increase in alkaline phosphatase levels that peaked between 300-400 U/L within 2-4 hours post transfusion. Normal levels of alkaline phosphatase fall in the range of 0-108 U/L. Significantly high levels of bilirubin were not obtained in all of the calves tested, however, several calves experienced a signficant increase above the normal range of 0-1.4 mg/dl.

Overall, four calves expired upon administration of the sensitization dose, one calf did not receive medication (Calf 12) and 3 calves did receive medication (Calves 5, 6, and 8). A blood analysis was not performed for calf 12 because death occurred at 4 minutes post transfusion. Calf 5 was treated with a 1:25 dilution prior to transfusion, and blood was collected for analysis until 5 hours post transfusion. A marked increase in alkaline phosphatase levels was noted, however, a significant change in bilirubin levels could not be detected in this short time period. Calf 6 was treated with a 1:125 dilution prior to transfusion, and blood was collected for analysis until 4 hours post transfusion. There was a significant increase in the levels of alkaline phosphatase and hemoglobin, suggesting hemolysis of the transfused erythrocytes. A blood analysis was not performed for calf 8 because death occurred 12 minutes post transfusion.

Two calves died within an hour of receiving the shock dose without medication prior to transfusion (Calves 2, and 3). Calf 2 was initially given the shock dose, however the transfusion was stopped after 250 ml. The calf survived, and blood was collected for analysis until 46 hours post transfusion. There was no significant increase in alkaline phosphatase levels, however, there was a marked increase in bilirubin by 9 hours post transfusion. A final shock dose was given 16 days later and blood was not collected because the calf died 15 minutes post transfusion. Calf 3 was given no medication prior to the shock dose, and died 15 minutes post transfusion.

Four calves expired within an hour of receiving the shock dose with medication prior to and during the transfusion (Calves 9, 10, 11, and 13). Blood was not collected for these calves after the shock dose because death occurred within 20 minutes post transfusion.

Three calves survived for at least 3 days after receiving the shock dose with medication (Calves 4, 7, and 14). Calf 4 received a 1:125 dilution prior to transfusion, and blood was collected until 44 hours post transfusion. There was a significant increase in the alkaline phosphatase level by 4.25 hours post transfusion, and a significant increase in the hemoglobin level by 2 hours post transfusion. Calf 7 received a 1:125 dilution prior to administration of the shock dose, and blood was collected until 30 hours post transfusion. There was a significant increase in alkaline phosphatase by 2.2 hours post transfusion, and a significant increase in the hemoglobin level by 25 minutes post transfusion. Both calf 4 and 7 died 3 days post transfusion, however medication was not continued in the days following the transfusion. Interestingly, the bilirubin level increased only slightly for calf 4, falling 0.3 mg/dl above the normal range, and did not fall outside the normal range for calf 7. Calf 14 received a #3 prior to administration of the shock dose, and no blood was collected. This calf experienced similar clinical symptoms to calves 4 and 7, however medication was continued daily for 7 days post transfusion. The calf survived and was observed for one month with no adverse reactions.

Eleven calves either died during the sensitization dose or shortly after the second transfusion. Yet only 3 calves receiving the second transfusion survived for at least three days, with one of those surviving at least a month. When compared to the results of the rabbit model, this would suggest that the calf is not the best model for studying the transfusion reaction. Perhaps this is due to the age of the animal as the calf model seems more susceptible to adverse reactions upon administration of the first transfusion. Analysis of these results indicates that there exists a need for post-transfusion treatment as the calves that were subjected to post-transfusion treatment had a better prognosis.

Overall, the increased alkaline phosphatase and hemoglobin levels indicate that there was hemolysis of the transfused equine erythrocytes in both treated and untreated animals. Hemoglobin levels suggest that this occurred within the first 1-2 hours post transfusion. Interestingly, the blood analysis suggests that treated calves administered the shock dose may experience lower levels of bilirubin than untreated calves.

### EXAMPLE 3

According to this example, the method of the invention was practiced to prevent rejection of a pancreatic beta-cell allograft transplant in rats. Specifically, a rat was pancreatectomized to obtain beta-cells which were treated and administered to other rats.

According to the test procedure, rats were anesthetize with ketamine (70 mg/kg) and xylazine (20 mg/kg). Shave belly. Weigh empty 15 ml conical tubes that the pancreata will be placed in (one for each rat). Surgery was performed to remove pancreas which was placed in a flat glass petri dish containing a thin layer of HBSS. Excess fat and any visible lymph nodes and blood clots were removed from pancreas and the cleaned pancreas was placed into clean glass crucible containing HBSS on ice. The pancreatic tissue was chopped into fine pieces as quickly as possible and excess fat was removed. The pancreas tissue was poured into a preweighed conical tube which was filled with HBSS and centrifuged at 1500 rpm for 1-1/2 minutes (enough to make a pellet of all of the tissue). The tissue was then dissolved with collagenase, washed and centrifuged and islet cells were isolated.

Five-fold and 1:25 dilutions of the beta-cells were made. Non-homogenized islet cells in HBSS were introduced intra-peritoneally to the recipient and immediately thereafter the recipient animal was subcutaneously treated with 0.2 cc of the appropriate dilution of homogenized cells BID as presented in Table 1, below.

TABLE 1

| Transplant | Islets | Therapy | Times/ | Donor | No. Days |
|---|---|---|---|---|---|
| No. | Injected | Dilution | Day | Strain | Alive Post. |
| 1 | 454 | 1:125 | 4 | bb | 36 |
| 2 | 186 | 1:25 | 4 | bb | 56 |
| 3 | 178 | 1:25 | 2 | bb | 20 |
| 4 | 467 | 0 | 0 | bb | 18 |
| 5 | 467 | 1:25 | 2 | bb | 4 |
| 6 | 434 | 0 | 0 | outbred | 21 |
| 7 | 400 | 1:25/1:125 | ½ | outbred | 28 |
| 8 | 600 | 1:125 | 2 | outbred | 21 |

### EXAMPLE 4

According to this example, four dogs were pancreatectomized and acted as transplant recipients. Without a pancreas they became immediately diabetic (no beta cells) with a projected survival time of 3-5 days without intervention. Four other dogs, unrelated to each other and the recipients were sacrificed, each pancreas removed, and the respective beta-cells isolated from each pancreas according to the general methods of Example 3.

An homogenate was prepared of beta-cells from each pancreas-four homogenates total and the recipients were given a beta-cell transplant from one of the unrelated donors (different donor for each recipient). The experiment was based upon the premise that successfully transplanted beta-cells could restore insulin production and rescue the diabetic dogs if those cells were not immunologically rejected. Historically, dogs receiving a placebo seldom survive more than 4 days post transplant.

Recipient dogs were also given, post-transplantation, twice daily subcutaneous injections of donor beta-cells in an attempt to block recipient rejection of the transplanted material and blood glucose levels are recorded on Table 2 below. The experimental dogs had a survival rate that was the same as that for dogs receiving no immunosuppressant with transplanted islet cells.

TABLE 2
Blood Glucose Levels

Subject

| | | 1 | 2 | 3 | 4 |
|---|---|---|---|---|---|
| Day | 0 | 114 | 104 | 99 | 86 |
| | 1 | 451 | 341 | 354 | 247 |
| | 2 | 450 | 356 | 401 | 247 |
| | 3 | 457 | 399 | 379 | 291 |
| | 4 | 570 | 343 | 385 | 374 |
| | 5 | Dead | Dead | 377 | 278 |
| | 6 | | | Dead | 336 |
| | 7 | | | | 365 |
| | 8 | | | | 326 |
| | 9 | | | | 326 |
| | 10 | | | | ? |
| | 11 | | | | Dead |

### EXAMPLE 5

According to this example, a donor skin tissue extract was tested as a therapeutic agent to prevent rejection of a skin allograft. A total of 35 rats were used. The medicated groups treated with 1:5, 1:625, 1:1325 dilutions of the tissue extract and a saline control group each had 3 animals. The control autograft and control allograft totaled 11 and 12 animals, respectively. After tissue grafting procedure, all animals were examined for graft rejection and bandages changed daily. A 0.2 cc subcutaneous injection of the therapeutic agent or control was given daily.

Evaluation of the antigenic preparation showed that the 1:1325 dilution had the only partial acceptance out of the three tested, 33.3%. The grafts receiving medicine 1:625 lasted the longest at 13 days. In comparison, the results of a previous study showed that the 1:5 dilution of the antigenic preparation had the best overall acceptance at 60% with n=5 and may indicate the lower dilution has greater utility in preventing rejection.

Numerous modifications and variations in the practice of the invention are expected to occur to those skilled in the art upon consideration of the presently preferred embodiments thereof. Consequently, the only limitations which should be placed upon the scope of the invention are those which appear in the appended claims.

What is claimed is:

1. A method of preventing allograft rejection in a transplant recipient comprising the step of administering to the recipient an antigenic preparation presenting antigens characteristic of the allograft in an amount effective to neutralize the immune response to the allograft.

2. The method of claim 1 wherein the antigenic preparation comprises donor tissue.

3. The method of claim 1 wherein from 10⁻⁸ to 10³ grams of antigenic material per dose is administered.

4. The method of claim 1 wherein from 10⁻⁴ to 10⁻⁴ grams of antigenic material per dose is administered.

5. The method of claim 1 wherein the antigenic preparation is administered by means selected from the group consisting of intravenous injection, intramuscular injection, subcutaneous injection, sublingual administration and oral administration.

6. The method of claim 1 wherein the antigenic preparation is administered during the transplantation event.

7. The method of claim 1 wherein the antigenic preparation is administered within an hour prior to the transplantation event.

8. The method of claim 1 wherein the antigenic preparation is administered after the transplantation event.

9. The method of claim 1 which is performed in combination with administration of additional immunosuppressive therapy.

10. The method of claim 1 wherein the allograft is a skin graft.

11. The method of claim 1 wherein the allograft is of pancreatic beta-cells.

12. The method of claim 1 wherein the allograft is a transfusion of blood or serum.
